(19) United States
(12) Patent Application Publication
(10) Pub. No.: US 2025/0281446 A1
(43) Pub. Date: Sep. 11, 2025

(54) COMPOSITIONS, SYSTEMS, AND METHODS FOR TREATING CANCER IN STANDARD OF CARE AND/OR PROFIBROTIC CHEMOTHERAPEUTIC AGENT-NAÏVE PATIENTS USING TUMOR TREATING FIELDS AND CHEMOTHERAPEUTIC AGENTS

(71) Applicant: Novocure GmbH, Baar (CH)

(72) Inventors: Gitit LAVY-SHAHAF, Haifa (IL); Tali VOLOSHIN-SELA, Haifa (IL); Lilach AVIGDOR, Haifa (IL)

(21) Appl. No.: 19/071,198

(22) Filed: Mar. 5, 2025

Related U.S. Application Data
(60) Provisional application No. 63/561,607, filed on Mar. 5, 2024.

(51) Int. Cl. A61K 31/337; A61K 31/404; A61K 31/436; A61K 31/44; A61K 31/495; A61K 31/496; A61K 31/506; A61K 31/5377; A61K 45/06; A61N 1/32; A61P 35/00

(52) U.S. Cl. CPC A61K 31/337; A61K 31/404; A61K 31/436; A61K 31/44; A61K 31/495; A61K 31/496; A61K 31/506; A61K 31/5377; A61K 45/06; A61N 1/32; A61P 35/00

(57) ABSTRACT

Compositions, systems, and methods for and treating cancer, as well as preventing an increase of volume of a tumor present in a body of a living subject, are disclosed. The systems and methods involve selection of a subject that is naïve for at least one standard-of-care chemotherapeutic drug (such as, but not limited to, a fibrotic-inducing chemotherapeutic agent), followed by application to the selected subject of an alternating electric field. The method may further include concurrent administration of at least one chemotherapeutic agent to the subject.

### CROSS REFERENCE TO RELATED APPLICATIONS

This application claims benefit under 35 USC § 119 (e) of U.S. Provisional Application No. 63/561,607, filed Mar. 5, 2024. The entire contents of the above-referenced patent application(s) are hereby expressly incorporated herein by reference.

### STATEMENT REGARDING FEDERALLY SPONSORED RESEARCH

Not Applicable.

### BACKGROUND

Tumor Treating Fields (TTFields) are low intensity (e.g., 1-3 V/cm) alternating electric fields within the intermediate frequency range (such as, but not limited to, 100-500 kHz) that target solid tumors by disrupting mitosis. This non-invasive treatment targets solid tumors and is described, for example, in U.S. Pat. Nos. 7,016,725; 7,089,054; 7,333,852; 7,565,205; 8,244,345; 8,715,203; 8,764,675; 10,188,851; and 10,441,776. TTFields are typically delivered through two pairs of transducer arrays that generate perpendicular fields within the treated tumor; the electrode arrays that make up each of these pairs are positioned on opposite sides of the body part that is being treated. More specifically, for the OPTUNE® system, one pair of electrodes is located to the left and right (LR) of the tumor, and the other pair of electrodes is located anterior and posterior (AP) to the tumor. TTFields are approved for the treatment of glioblastoma multiforme (GBM), and may be delivered, for example, via the OPTUNE® system (Novocure Limited, St. Helier, Jersey), which includes transducer arrays placed on the patient's shaved head.

Each transducer array used for the delivery of TTFields in the OPTUNE® device comprises a set of ceramic disk electrodes, which are coupled to the patient's skin (such as, but not limited to, the patient's shaved head for treatment of GBM) through a layer of conductive medical gel. The purpose of the medical gel is to deform to match the body's contours and to provide good electrical contact between the arrays and the skin; as such, the gel interface bridges the skin and reduces interference. The device is intended to be continuously worn by the patient for 2-4 days before removal for hygienic care and re-shaving (if necessary), followed by reapplication with a new set of arrays. As such, the medical gel remains in substantially continuous contact with an area of the patient's skin for a period of 2-4 days at a time. In addition, the arrays can be shifted a few centimeters in either direction to allow the skin to heal from one period of treatment to the next. Therefore, a portion of skin that was covered by electrodes/gel for a 2-4 day period could then be uncovered for 2-4 days when the replaced electrodes are shifted slightly; then the device may be reapplied to the original portion of skin for the next 2-4 day period.

TTFields have been studied clinically together with standard-of-care (SOC) immunotherapy or chemotherapy regimens for treatment of non-small cell lung cancer that has progressed on platinum-based therapy (Leal, et al. (2023) Lancet Oncol, 24 (9): 1002-1017) and preclinically together with immune checkpoint inhibitors (Voloshin, et al. (2020) Cancer Immunol Immunother, 69 (7): 1191-1204; Barsheshet, et al. (2022) Int J Mol Sci, 23 (22): 14073).

### BRIEF DESCRIPTION OF THE DRAWINGS

FIG. 1 graphically depicts a study design that was utilized to evaluate the safety and efficacy of TTFields therapy concomitant with weekly paclitaxel compared to weekly paclitaxel alone in Platinum-Resistant Ovarian Cancer (PROC). Abbreviations/Acronyms utilized: CT, computerized tomography; ECOG, Eastern Cooperative Oncology Group; HR, hazard ratio; HRQOL, health-related quality of life; MRI, magnetic resonance imaging; ORR, objective response rate; OS, overall survival; PFS, progression-free survival; PROC, platinum-resistant ovarian cancer; TTFields, Tumor Treating Fields; QW, every week; Q4W, every 4 weeks; Q8W, every 8 weeks.

FIG. 2 contains a table of the demographics and baseline characteristics of the study group of FIG. 1. *PLD represented 93% of the anthracyclines used, and the remaining 7% a non-specified doxorubicin formulation. Abbreviations/acronyms used: BRCA, breast cancer gene; ECOG PS, Eastern Cooperative Oncology Group Performance Status score; PLD, pegylated liposomal doxorubicin; ITT, intention-to-treat; TTFields, Tumor Treating Fields.

FIG. 3 graphically depicts the overall survival in the ITT population of the study.

FIG. 4 provides a summary of subgroup analyses of the study participants according to stratification factors and exploratory analyses. *PLD represented 93% of the anthracyclines used, and the remaining 7% a non-specified doxorubicin formulation. BRCA, breast cancer gene; PARP, poly-ADP ribose polymerase; PLD, pegylated liposomal doxorubicin.

FIG. 5 illustrates a multivariant analyses of the study participants. Model Parameters: Arm, Arm X prior PLD* use, prior bevacizumab, BRCA status, number of line following platinum, total number of prior lines, prior PARP, FIGO stage, grade, ascites, platinum free interval, patient age, and ECOG. Indicating variables maintained in the model defined by significance of 0.25 for variables exclusion. *PLD represented 93% of the anthracyclines used, and the remaining 7% a non-specified doxorubicin formulation. †The mITT population includes all patients who received ≥1 complete 28-day cycle of study treatment. BRCA, breast cancer gene; ECOG, Eastern Cooperative Oncology Group; FIGO, International Federation of Gynaecology and Obstetrics; mITT, modified intention-to-treat; PARP, poly-ADP ribose polymerase; PH, proportional hazard; PLD, pegylated liposomal doxorubicin; TTFields, Tumor Treating Fields.

FIG. 6 contains a table of the demographics and baseline characteristics of the PLD*-naïve subgroup. *PLD represented 93% of the anthracyclines used, and the remaining 7% a non-specified doxorubicin formulation. BRCA, breast cancer gene; ECOG PS, Eastern Cooperative Oncology Group Performance Status score; ITT, intention-to-treat; PLD, pegylated liposomal doxorubicin; TTFields, Tumor Treating Fields.

FIG. 7 graphically depicts the overall survival in the PLD*-naïve subgroup. *PLD represented 93% of the anthracyclines used, and the remaining 7% a non-specified doxorubicin formulation. HR, hazard ratio; OS, overall survival; PLD, pegylated liposomal doxorubicin; TTFields, Tumor Treating Fields.

FIG. 8 contains a table analyzing the safety of the study with respect to adverse events (AEs). PLD, pegylated liposomal doxorubicin; SAE, serious adverse event; TTFields, Tumor Treating Fields.

FIG. 9 contains a table analyzing the safety of the study with respect to AEs related to TTFields therapy. AE, adverse event; SAE, serious adverse event; TTFields, Tumor Treating Fields.

FIG. 10 graphically depicts analysis of overall survival in groups treated with Paclitaxel alone or TTFields plus Paclitaxel and comparing doxorubicin-naïve patients to patients that had received doxorubicin prior to use.

FIG. 11 graphically depicts analysis of overall survival of doxorubicin-naïve patients with treatment with Paclitaxel alone or TTFields plus Paclitaxel.

FIG. 12 illustrates a multivariate analysis for the study of FIGS. 10-11.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference

All of the compositions, assemblies, systems, kits, and/or methods disclosed herein can be made and executed without undue experimentation in light of the present disclosure. While the compositions, assemblies, systems, kits, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (e.g., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “patient” or “subject” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent/element/method to a patient for therapeutic and/or prophylactic/preventative purposes.

The term “therapeutic composition” or “pharmaceutical composition” as used herein refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof, or an amount of a treatment protocol (e.g., an alternating electric field), sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of at least one condition, disease, and/or infection. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “concomitant therapy” and “adjunct therapy,” and will be understood to mean that the patient in need of treatment is treated or given another drug for the condition/disease/infection in conjunction with the treatments of the present disclosure. This concurrent therapy can be sequential therapy, where the patient is treated first with one treatment protocol/pharmaceutical composition and then the other treatment protocol/pharmaceutical composition, or the two treatment protocols/pharmaceutical compositions are given simultaneously.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, intratumoral, intertumoral, and/or intravenous routes, and including both local and systemic applications. In addition, the compositions of the present disclosure (and/or the methods of administration of same) may be designed to provide delayed, controlled, or sustained release using formulation techniques which are well known in the art.

Turning now to the inventive concept(s), it has been found that the use of a standard-of-care and/or profibrotic chemotherapeutic agent (or other profibrotic antitumor treatment) prior to beginning alternating electric fields (e.g., TTFields) treatment leads to fibrosis in tumors, and this fibrosis decreases the conductivity of the tumors, thereby reducing the effectiveness of treatment with alternating electric fields. Therefore, in the present disclosure, TTFields cancer treatment is targeted to subjects that are naïve for standard-of-care and/or profibrotic chemotherapies (and in certain non-limiting embodiments, when profibrotic chemotherapies, other profibrotic antitumor treatments as well). In certain non-limiting embodiments, the cancer treatment may comprise a concurrent therapy that includes the use of alternating electric fields (e.g., TTFields) in addition with at least one chemotherapeutic agent. In yet further non-limiting embodiments, the cancer treatment may comprise a concurrent therapy that includes the use of alternating electric fields (e.g., TTFields) in combination with at least one chemotherapeutic agent and at least one anti-fibrotic agent. The present disclosure demonstrates that standard-of-care chemotherapy-naïve subjects and/or profibrotic chemotherapy/profibrotic antitumor treatment-naïve subjects treated with TTFields therapy exhibited statistically significant improved survival rates when compared to TTFields-treated subjects that had previously been exposed to a standard-of-care chemotherapy and/or profibrotic chemotherapeutic agent/profibrotic antitumor treatment. In addition, this statistically improved survival rate is observed when concurrent therapy of TTFields plus chemotherapy agent(s) (including profibrotic agent(s)) is administered to the standard-of-care chemotherapy-naïve subjects and/or profibrotic chemotherapy/profibrotic antitumor treatment-naïve subjects.

Table 1 lists various cancers and the current first line standard-of-care chemotherapies therefor.

TABLE 1

| Cancer | First Line Standard of Care Drugs |
|---|---|
| Glioblastoma | temozolomide (TMZ), bevacizumab, carmustine |
| Mesothelioma | bevacizumab, carboplatin, cisplatin, cyclophophamide, |
| | doxorubicin, gemcitabine, ipilimumab, nivolumab, oxaliplatin, |
| | pembrolizumab, pemetrexed, raltitrexed, vinorelbine |
| Ovarian Cancer | docetaxel, gemcitabine, paclitaxel, PARP Inhibitors |
| Pancreatic Cancer | capecitabine, fluorouracil, folfirinox, gemcitabine, irinotecan, |
| | leucovorin, nalirifox, oxaliplatin, paclitaxel, pemetrexed |
| Non-Small Cell Lung | carboplatin, cisplatin, docetaxel, etoposide, gemcitabine, |
| | paclitaxel, pemetrexed, vinorelbine |

Certain additional non-limiting embodiments of the present disclosure are directed to a method of treating cancer in a subject. The method includes the steps of: (1) selecting a subject that is naïve for (a) standard-of-care chemotherapy and/or (b) profibrotic chemotherapy and profibrotic antitumor treatment; (2) applying an alternating electric field to a target region of the subject for a period of time; and (3) administering at least one composition to the subject, wherein the at least one composition comprises at least one chemotherapeutic agent.

Certain additional non-limiting embodiments of the present disclosure are directed to a method of reducing a volume of a tumor present in a body of a living subject, wherein the tumor includes a plurality of cancer cells. The method includes the steps of: (1) selecting a subject that is naïve for (a) standard-of-care chemotherapy and/or (b) profibrotic chemotherapy and profibrotic antitumor treatment; (2) applying an alternating electric field to a target region of the subject for a period of time; and (3) administering at least one composition to the subject, wherein the at least one composition comprises at least one chemotherapeutic agent.

Certain additional non-limiting embodiments of the present disclosure are directed to a method of preventing an increase of volume of a tumor, wherein the tumor is present in a body of a living subject and includes a plurality of cancer cells. The method includes the steps of: (1) selecting a subject that is naïve for (a) standard-of-care chemotherapy and/or (b) profibrotic chemotherapy and profibrotic antitumor treatment (such as, but not limited to, radiation); (2) applying an alternating electric field to a target region of the subject for a period of time; and (3) administering at least one composition to the subject, wherein the at least one composition comprises at least one chemotherapeutic agent.

In any of the above-described methods, the subject may have glioblastoma, and the subject is naïve for standard-of-care chemotherapy selected from the group consisting of temozolomide (TMZ), bevacizumab, carmustine, and combinations thereof. In a particular (but non-limiting) embodiment, the subject is naïve for TMZ. In another particular (but non-limiting) embodiment, the subject is naïve for at least two or all of temozolomide (TMZ), carmustine, and bevacizumab.

In any of the above-described methods, the subject may have mesothelioma, and the subject is naïve for standard-of-care chemotherapy selected from the group consisting of bevacizumab, carboplatin, cisplatin, cyclophophamide, doxorubicin, gemcitabine, ipilimumab, nivolumab, oxaliplatin, pembrolizumab, pemetrexed, raltitrexed, vinorelbine, and combinations thereof. In another particular (but non-limiting) embodiment, the subject is naïve for at least two, at least three, at least four, at least five, at least six, at least seven, at least eight, at least nine, at least ten, at least eleven, at least twelve, or all of bevacizumab, carboplatin, cisplatin, cyclophophamide, doxorubicin, gemcitabine, ipilimumab, nivolumab, oxaliplatin, pembrolizumab, pemetrexed, raltitrexed, and vinorelbine.

In any of the above-described methods, the subject may have ovarian cancer, and the subject is naïve for standard-of-care chemotherapy selected from the group consisting of docetaxel, gemcitabine, paclitaxel, PARP Inhibitors, and combinations thereof. In another particular (but non-limiting) embodiment, the subject is naïve for at least two, at least three, or all of docetaxel, gemcitabine, paclitaxel, and PARP Inhibitors.

In any of the above-described methods, the subject may have pancreatic cancer, and the subject is naïve for standard-of-care chemotherapy selected from the group consisting of capecitabine, fluorouracil, folfirinox, gemcitabine, irinotecan, leucovorin, nalirifox, oxaliplatin, paclitaxel, pemetrexed, and combinations thereof. In another particular (but non-limiting) embodiment, the subject is naïve for at least two, at least three, at least four, at least five, at least six, at least seven, at least eight, at least nine, or all of capecitabine, fluorouracil, folfirinox, gemcitabine, irinotecan, leucovorin, nalirifox, oxaliplatin, paclitaxel, and pemetrexed.

In any of the above-described methods, the subject may have non-small cell lung cancer, and the subject is naïve for standard-of-care chemotherapy selected from the group consisting of carboplatin, cisplatin, docetaxel, etoposide, gemcitabine, paclitaxel, pemetrexed, vinorelbine, and combinations thereof. In another particular (but non-limiting) embodiment, the subject is naïve for at least two, at least three, at least four, at least five, at least six, at least seven, or all of carboplatin, cisplatin, docetaxel, etoposide, gemcitabine, paclitaxel, pemetrexed, and vinorelbine.

Certain additional non-limiting embodiments of the present disclosure are directed to a method of treating cancer in a subject. The method includes the steps of: (1) selecting a subject that is doxorubicin-naïve; (2) applying an alternating electric field to a target region of the subject for a period of time; and (3) administering at least one composition to the subject, wherein the at least one composition comprises at least one chemotherapeutic agent.

Certain additional non-limiting embodiments of the present disclosure are directed to a method of reducing a volume of a tumor present in a body of a living subject, wherein the tumor includes a plurality of cancer cells. The method includes the steps of: (1) selecting a subject that is doxorubicin-naïve; (2) applying an alternating electric field to a target region of the subject for a period of time; and (3) administering at least one composition to the subject, wherein the at least one composition comprises at least one chemotherapeutic agent.

Certain additional non-limiting embodiments of the present disclosure are directed to a method of preventing an increase of volume of a tumor, wherein the tumor is present in a body of a living subject and includes a plurality of cancer cells. The method includes the steps of: (1) selecting a subject that is doxorubicin-naïve; (2) applying an alternating electric field to a target region of the subject for a period of time; and (3) administering at least one composition to the subject, wherein the at least one composition comprises at least one chemotherapeutic agent.

The steps of any of the methods of the present disclosure may be performed concomitantly or serially, and in particular, substantially simultaneously or wholly or partially sequentially.

The methods of the present disclosure may be utilized to treat any types of cancer cells/cancers/tumors that respond to treatment with alternating electric fields (e.g., TTFields). Non-limiting examples of cancer cells/cancers/tumors that can be treated in accordance with the present disclosure include hepatocellular carcinoma/carcinoma cells, glioblastoma/glioblastoma cells, pleural mesothelioma/mesothelioma cells, differentiated thyroid cancer/cancer cells, advanced renal cell carcinoma/carcinoma cells, ovarian cancer/cancer cells, pancreatic cancer/cancer cells, lung cancer/cancer cells, breast cancer/cancer cells, colorectal cancer cells and the like, as well as any combination thereof.

In a certain particular (but non-limiting) embodiment, the cancer is in the form of a solid tumor.

Any type of conductive or non-conductive electrode(s) and/or transducer array(s) that can be utilized for generating an alternating electric field that are known in the art or otherwise contemplated herein may be utilized for generation of the alternating electric field in accordance with the methods of the present disclosure. Non-limiting examples of electrodes and transducer arrays that can be utilized for generating an alternating electric field in accordance with the present disclosure include those that function as part of a TTFields system as described, for example but not by way of limitation, in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016.

The alternating electric field may be generated at any frequency in accordance with the present disclosure. For example (but not by way of limitation), the alternating electric field may have a frequency of about 50 kHz, about 75 kHz, about 100 kHz, about 125 kHz, about 150 kHz, about 175 kHz, about 200 kHz, about 225 kHz, about 250 kHz, about 275 kHz, about 300 kHz, about 325 kHz, about 350 kHz, about 375 kHz, about 400 kHz, about 425 kHz, about 450 kHz, about 475 kHz, about 500 kHz, about 550 kHz, about 600 kHz, about 650 kHz, about 700 kHz, about 750 kHz, about 800 kHz, about 850 kHz, about 900 kHz, about 950 kHz, about 1 MHZ, about 2 MHZ, about 3 MHz, about 4 MHZ, about 5 MHz, about 6 MHZ, about 7 MHz, about 8 MHz, about 9 MHz, about 10 MHZ, and the like, as well as a range formed from any of the above values (e.g., a range of from about 50 kHz to about 10 MHz, a range of from about 50 kHz to about 1 MHz, a range of from about 50 kHz to about 500 kHz, a range of from about 100 kHz to about 500 kHz, a range of from about 150 kHz to about 300 kHz, etc.), and a range that combines two integers that fall between two of the above-referenced values (e.g., a range of from about 122 kHz to about 313 kHz, a range of from about 78 kHz to about 298 kHz, etc.).

In certain particular (but non-limiting) embodiments, the alternating electric field may be imposed at two or more different frequencies. When two or more frequencies are present, each frequency is selected from any of the above-referenced values, or a range formed from any of the above-referenced values, or a range that combines two integers that fall between two of the above-referenced values.

The alternating electric field may have any field strength in the subject/cancer cells, so long as the alternating electric field is capable of functioning in accordance with the present disclosure. For example (but not by way of limitation), the alternating electric field may have a field strength of at least about 1 V/cm, about 1.5 V/cm, about 2 V/cm, about 2.5 V/cm, about 3 V/cm, about 3.5 V/cm, about 4 V/cm, about 4.5 V/cm, about 5 V/cm, about 5.5 V/cm, about 6 V/cm, about 6.5 V/cm, about 7 V/cm, about 7.5 V/cm, about 8 V/cm, about 9 V/cm, about 9.5 V/cm, about 10 V/cm, about 10.5 V/cm, about 11 V/cm, about 11.5 V/cm, about 12 V/cm, about 12.5 V/cm, about 13 V/cm, about 13.5 V/cm, about 14 V/cm, about 14.5 V/cm, about 15 V/cm, about 15.5 V/cm, about 16 V/cm, about 16.5 V/cm, about 17 V/cm, about 17.5 V/cm, about 18 V/cm, about 18.5 V/cm, about 19 V/cm, about 19.5 V/cm, about 20 V/cm, and the like, as well as a range formed from any of the above values (e.g., a range of from about 1 V/cm to about 20 V/cm, a range of from about 1 V/cm to about 10 V/cm, a range of from about 1 V/cm to about 4 V/cm, etc.), and a range that combines two integers that fall between two of the above-referenced values (e.g., a range of from about 1.1 V/cm to about 18.6 V/cm, a range of from about 1.2 V/cm to about 9.8 V/cm, a range of from about 1.3 V/cm to about 4.7 V/cm, etc.).

The alternating electric field may be applied in a single direction between a pair of arrays or may be alternating in two or more directions/channels between two or more pairs of arrays (e.g., front-back and left-right). For example, certain TTFields devices (such as, but not limited to, the OPTUNE® system (Novocure Limited, St. Helier, Jersey)) operate in two directions in order to increase the chances that a dividing cell will be aligned with the electric field such that the electric field can have the desired anti-mitotic effect. However, it will be understood that the scope of the present disclosure also includes the application of the alternating electric field in a single direction. The term “alternating electric field” as used herein will be understood to include application in a single direction/channel as well as in two or more directions/channels; in addition, the term “alternating electric field” as used herein will be understood to include both application of a single alternating electric field as well as application of a plurality of alternating electric fields in succession for a duration of time.

The alternating electric field may be applied for any continuous or cumulative period of time sufficient to achieve a reduction in viability of cancer cells and/or a reduction in tumor volume (and/or a prevention of increase in tumor volume). The period of time that the alternating electric field is applied includes both a continuous period of time as well as a cumulative period of time. That is, the period of time that the alternating electric field is applied includes a single session (i.e., continuous application) as well as multiple sessions with minor breaks in between sessions (i.e., consecutive application for a cumulative period). For example, a subject is allowed to take breaks during treatment with an alternating electric field device and is only expected to have the device positioned on the body and operational for at least about 50%, at least about 60%, at least about 70%, or at least about 80% of the total treatment period (e.g., over a course of one day, one week, two weeks, one month, two months, three months, four months, five months, etc.).

For example, but not by way of limitation, the alternating electric field may be applied for a continuous or cumulative period of time of at least about 1 hour, about 2 hours, about 3 hours, about 4 hours, about 5 hours, about 6 hours, about 7 hours, about 8 hours, about 9 hours, about 10 hours, about 11 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, about 8 days, about 9 days, about 10 days, about 11 days, about 12 days, about 13 days, about 14 days, about 21 days, about 1 month, about 2 months, about 3 months, about 4 months, about 5 months, about 6 months, and the like, as well as a range formed from any of the above values (e.g., a range of from about 1 hour to about 6 months, a range of from about 24 hours to about 72 hours, etc.), and a range that combines two integers that fall between two of the above-referenced values (e.g., a range of from about 14 hours to about 68 hours, etc.).

In a particular (but non-limiting) embodiment, the period of time that the alternating electric field is applied is at least about 24 cumulative hours within 48 consecutive hours.

Any compositions that function as chemotherapeutic agents that are known in the art or are otherwise contemplated herein may be utilized in accordance with the present disclosure, so long as the compositions are capable of functioning as described herein. Non-limiting examples include anti-fibrotic (or likely anti-fibrotic) chemotherapeutic agents such as (but not limited to) a taxane (such as, but not limited to, paclitaxel, docetaxel, cabazitaxel, and abraxane), nimustine, gefitinib, pazopanib, nintedanib, sorafenib, sunitinib, imatinib, everolimus, and the like, as well as combinations thereof. In addition, chemotherapeutic agents that induce (or likely induce) fibrosis in a tumor may also be used in accordance with the present disclosure. Non-limiting examples of profibrotic (or likely profibrotic) chemotherapeutic agents include anthracyclines (such as, but not limited to, Daunorubicin, Doxorubicin, Epirubicin, Idarubicin, Mitotaxantrone, Valrubicin), bleomycin, carmustine, cisplatin, oxaliplatin, gemcitabine, cyclophosphamide, fluorouracil, leucovorin, capecitabine, mitoxantrone, bevacizumab, carboplatin, olaparib, dacomitinib, raltitrexed, and the like, as well as combinations thereof. Also, chemotherapeutic agents that have not previously been demonstrated to be profibrotic may be utilized; non-limiting examples thereof include temozolomide, docetaxel, lomustine, pemetrexed, vinorelbine, irinotecan, etoposide, afatinib, osimertinib, erlotinib, pazopanib, sotorasinib, ramucirumab, mirvetuximab, necitumumab, niraparib, durvalumab, atezolizumab, cemiplimab, avelumab, and the like, as well as combinations thereof. In addition, compositions that contain any combination of one or more anti-fibrotic (or likely anti-fibrotic) chemotherapeutic agents, one or more profibrotic (or likely profibrotic) chemotherapeutic agents, and/or one or more chemotherapeutic agents that have not been shown to have any effect on fibrosis may also be utilized in accordance with the present disclosure.

In a particular (but non-limiting) embodiment, the patient has ovarian cancer, and the at least one chemotherapeutic agent administered to the patient comprises paclitaxel.

As disclosed herein above, it is known in the art that certain chemotherapeutic agents are both anti-tumorigenic as well as anti-fibrotic. Therefore, the chemotherapeutic composition administered to the subject may include combinations of two or more chemotherapeutic agents, where at least one of the chemotherapeutic agents present is anti-fibrotic (or likely anti-fibrotic), while another chemotherapeutic agent may be present that is profibrotic (or likely profibrotic).

Alternatively (and/or in addition thereto), in certain non-limiting embodiments, an anti-fibrotic agent may also be administered to the subject. The use of anti-fibrotic agents may be particularly useful when the chemotherapeutic agent utilized is profibrotic (or likely profibrotic), and/or when the chemotherapeutic agent is not anti-fibrotic (or wherein the ability to induce or reduce fibrosis is not known); however, anti-fibrotic agents may also be utilized in combination with chemotherapeutic agents that are anti-fibrotic (or likely anti-fibrotic). Any compositions that function as anti-fibrotic agents that are known in the art or are otherwise contemplated herein may be utilized in accordance with the present disclosure, so long as the compositions are capable of functioning as described herein.

Various classes of compounds that are known to be anti-fibrotic can be utilized as anti-fibrotic agents in accordance with the present disclosure. Non-limiting examples of such classes of compounds include calcium channel blockers, angiotensin II receptor blockers (ARBs), IL11 inhibitors (e.g., IL11 antagonists or IL11 neutralizing antibodies), IL13 inhibitors (e.g., IL13 antagonists or IL13 neutralizing antibodies), receptor tyrosine kinase inhibitors (RTKI), rennin-angiotensin-aldosterone system (RAAS) inhibitors, angiotensin-converting enzyme (ACE) inhibitors, anti-hypertension agents, and the like, as well as any combinations thereof.

Particular (but non-limiting) specific examples of anti-fibrotic agents that may be utilized in accordance with the present disclosure include losartan, fasudil, pirfenidone, pintedanib, nintedanib, hyaluronidase, tranilast, vismodegib, felodipine, verapamil, diltiazem, nifedipine, and the like, as well as any combinations thereof.

The composition(s) of the present disclosure may be provided with any formulation known in the art or otherwise contemplated herein. In certain particular (but non-limiting) embodiments, the compositions contain one or more pharmaceutically acceptable carriers (and as such, the composition may also be referred to as a “pharmaceutical composition”). Non-limiting examples of suitable pharmaceutically acceptable carriers include water; saline; dextrose solutions; fructose or mannitol; calcium carbonate; cellulose; ethanol; oils of animal, vegetative, or synthetic origin; carbohydrates, such as glucose, sucrose, or dextrans; antioxidants, such as ascorbic acid or glutathione; chelating agents; low molecular weight proteins; detergents; liposomal carriers; nanocarriers; scaffolds that allowed delayed drug release (such as, but not limited to, hydrogels); buffered solutions, such as sodium chloride, saline, phosphate-buffered saline, and/or other substances which are physiologically acceptable and/or safe for use; diluents; excipients such as polyethylene glycol (PEG); or any combination thereof. Suitable pharmaceutically acceptable carriers for pharmaceutical formulations are described, for example, in Remington: The Science and Practice of Pharmacy, 23rd ed. (2020).

In certain particular (but non-limiting) embodiments, the composition(s) of the present disclosure may further contain one or more additional active agents. Various active agents that can be utilized concurrently with alternating electric fields and/or immunomodulatory cytokine inhibitors are known in the art, and certain combination therapies are approved by the FDA or currently in clinical trials testing.

In addition, any of the compositions of the present disclosure may contain other agents that allow for administration of the compositions via a particular administration route. For example, but not by way of limitation, the compositions may be formulated for administration by oral, topical, transdermal, parenteral, subcutaneous, intranasal, mucosal, intramuscular, intraperitoneal, intravitreal, intratumoral, intertumoral, and/or intravenous routes. Based on the route of administration, the compositions may also contain one or more additional components in addition to the active agent(s) (e.g., immunomodulatory cytokine inhibitor(s) and/or additional therapeutic agent(s)). Examples of additional secondary compounds that may be present include, but are not limited to, fillers, salts, buffers, preservatives, stabilizers, solubilizers, wetting agents, emulsifying agents, dispersing agents, gels, adhesives, and other materials well known in the art.

In a particular (but non-limiting) embodiment, any of the compositions of the present disclosure is administered via injection or implantation into the subject. For example (but not by way of limitation), in some instances, it may be desired that the composition(s) be administered on a local/regional level to ensure targeting of the composition(s) to a specific location in the body of the subject and inhibit non-specific interactions in other parts of the body; in other instances, a more systemic administration may be desired.

Any of the compositions of the present disclosure may be administered before or after application of the alternating electric field has begun. In certain particular (but non-limiting) embodiments, at least one composition may be administered before the application of the alternating electric field has begun. In other particular (but non-limiting) embodiments, the at least one composition may be administered after the application of the alternating electric field has begun. In particular (but not by way of limitation), the composition(s) may be administered during application of the alternating electric field (e.g., before the period of time that the alternating electric field is applied has elapsed) and/or after application of the alternating electric field has elapsed.

For example (but not by way of limitation), any of the compositions of the present disclosure may be administered after application of the alternating electric field has commenced by a period of at least about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, as well as a range formed from any of the above values (e.g., a range of from about 24 hours to about 96 hours, etc.), and a range that combines two integers that fall between two of the above-referenced values (e.g., a range of from about 14 hours to about 94 hours, etc.). In a particular (but non-limiting) embodiment, the at least one composition is administered at least about 24 hours after application of the alternating electric field has begun.

In other non-limiting examples, any of the compositions of the present disclosure may be administered after the period of time that the alternating electric field is applied has elapsed, wherein the one or more composition(s) is administered within about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, of when the period of time elapsed.

In a particular (but non-limiting) embodiment, one or more of the compositions of the present disclosure is administered within about 96 hours of when the period of time elapsed.

The compositions of the present disclosure may be administered to the cancer cells/subject at any concentration that provides a therapeutically effective concentration of the active agent(s) (i.e., chemotherapeutic agent(s) and/or anti-fibrotic agent(s)). In certain non-limiting embodiments, the application of the alternating electric field reduces the amount of the active agent required to be therapeutically effective when compared to a normal therapeutically effective amount of active agent administered in the absence of an alternating electric field. For example, but not by way of limitation, the therapeutically effective concentration of the composition may be reduced by at least about 25%, about 30%, about 35%, about 40%, about 45%, about 50%, about 55%, about 60%, about 65%, about 70%, about 75% or more with respect to a dosage of the composition known to be therapeutically effective in the absence of application of an alternating electric field. In a particular (but non-limiting) embodiment, the therapeutically effective concentration of the composition is reduced by at least about 50% when compared to a dosage of the composition known to be therapeutically effective in the absence of an alternating electric field.

The therapeutically effective concentration of each active agent utilized in accordance with the present disclosure may be, for example (but not by way of limitation), about 1 nM, about 10 nM, about 20 nM, about 30 nM, about 40 nM, about 50 nM, about 60 nM, about 70 nM, about 80 nM, about 90 nM, about 100 nM, about 125 nM, about 150 nM, about 175 nM, about 200 nM, about 250 nM, about 300 nM, about 350 nM, about 400 nM, about 450 nM, about 500 nM, about 550 nM, about 600 nM, about 650 nM, about 700 nM, about 750 nM, about 800 nM, about 850 nM, about 900 nM, about 950 nM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, about 50 mM, and the like, as well as a range formed from any of the above values (e.g., a range of from about 12.5 nM to about 100 nM, a range of from about 1 mM to about 20 mM, etc.), and a range that combines two integers that fall between two of the above-referenced values (e.g., a range of from about 17 nM to about 83 nM, etc.).

In a particular (but non-limiting) embodiment, the therapeutically effective concentration of each active agent is from about 10 nM to about 100 nM.

In particular (but non-limiting) embodiments, the therapeutically effective concentration of each active agent utilized in accordance with the present disclosure may be, for example (but not by way of limitation), about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, and the like, as well as a range formed from any of the above values (e.g., a range of from about 10 mg/kg to about 50 mg/kg, a range of from about 1 mg/kg to about 40 mg/kg, a range of from about 1 mg/kg to about 30 mg/kg, a range of from about 1 mg/kg to about 25 mg/kg, a range of from about 1 mg/kg to about 20 mg/kg, a range of from about 1 mg/kg to about 10 mg/kg, etc.).

In certain particular (but non-limiting) embodiments, the method includes one or more additional steps. For example (but not by way of limitation), the method may further include the step of: discontinuing the application of the alternating electric field (such as, but not limited to, to allow the cells/tissue to recover). In addition, any of the steps may be repeated one or more times.

In certain particular (but non-limiting) embodiments, any of the compositions of the present disclosure may be administered by any dosage regimen known in the art. For example, but not by way of limitation, each composition may be administered in a single dosage or multiple dosages over a defined treatment period. For example (but not by way of limitation), a therapeutically effective concentration of one or more compositions may be administered about once every 4 hours, about once every 8 hours, about once every 12 hours, about once every day, about once every other day, about once every three days, about once a week, about twice a week, about three times a week, about once every two weeks, about once every three weeks, about once a month, and the like, as well as a range formed from any of the above values (a range of about once every 4 to 8 hours, a range of from about once a week to about once a month, etc.).

In addition, when multiple compositions are administered (i.e., two or more chemotherapeutic agents, two or more anti-fibrotic agents, and/or at least one chemotherapeutic agent and at least one anti-fibrotic agent, with at least two agents being present in different compositions), the two or more compositions may be administered via the same route (e.g., both administered intravenously), or the two or more compositions may be administered by different routes (e.g., one composition orally administered and another composition intravenously administered).

In certain particular (but non-limiting) embodiments, the method involves concurrent therapy with yet additional compositions. As such, the method may include an additional step of administering at least one additional therapeutic composition to the cancer cells/subject.

When present, administration of the at least one additional therapeutic composition may be performed substantially simultaneously or wholly or partially sequentially with the administration of any of the composition(s) containing the immunomodulatory cytokine inhibitor(s), whereby the separate compositions are administered simultaneously or wholly or partially sequentially. Also, when the method includes administration of the additional composition, the optional administration step may be performed before or after the application of the alternating electric field has begun, during application of the alternating electric field, and/or after application of the alternating electric field has elapsed, in the same manner(s) and time frame(s) as described above for the other composition(s).

In certain particular (but non-limiting) embodiments, the method may further comprise the step of administering at least one additional therapy to the cells/subject. Any therapies known in the art or otherwise contemplated herein for use with alternating electric fields (e.g., TTFields), chemotherapeutic agents, and/or anti-fibrotic agents may be utilized in accordance with the methods of the present disclosure. Non-limiting examples of additional therapies that may be utilized include radiation therapy, photodynamic therapy, transarterial chemoembolization (TACE), or combinations thereof.

Any of the method steps may be repeated one or more times. Each of the steps can be repeated as many times as necessary. When the step of applying the alternating electric field is repeated, the transducer arrays may be placed in slightly different positions on the subject than their original placement; relocation of the arrays in this manner may further aid in treatment of the tumor/cancer. In addition, any of the steps of administering any of the compositions/additional therapies may be repeated various times and at various intervals to follow any known and/or generally accepted dosage/treatment regimen for the composition(s)/therapy(ies).

Certain non-limiting embodiments of the present disclosure are related to kits that include any of the components of the alternating electric field (e.g., TTFields) generating systems disclosed or otherwise contemplated herein (such as, but not limited to, one or more transducer arrays and/or one or more hydrogel compositions, as disclosed in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016) in combination with at least one of any of the compositions disclosed or otherwise contemplated herein. The kits may optionally further include one or more of any of the optional compositions disclosed or otherwise contemplated herein (such as, but not limited to, one or more optional compositions containing at least one additional active agent). The kits may optionally further include one or more devices (or one or more components of devices) utilized in one or more additional therapy steps.

In a particular (but non-limiting) embodiment, the kit may further include instructions for performing any of the methods disclosed or otherwise contemplated herein. For example (but not by way of limitation), the kit may include instructions for applying one or more components of the alternating electric field (e.g., TTFields) generating device to the skin of the patient, instructions for applying the alternating electric field to the patient, instructions for formulating one or more of the compositions, instructions for when and how to administer the one or more compositions, and/or instructions for when to activate and turn off the alternating electric field in relation to the administration of the composition(s) and/or optional therapy steps.

In addition to the components described in detail herein above, the kits may further contain other component(s)/reagent(s) for performing any of the particular methods described or otherwise contemplated herein. For example (but not by way of limitation), the kits may additionally include: (i) components for preparing the skin prior to disposal of the hydrogel compositions and/or transducer arrays thereon (e.g., a razor, a cleansing composition or wipe/towel, etc.); (ii) components for removal of the gel/transducer array(s); (iii) components for cleansing of the skin after removal of the gel/transducer array(s); and/or (iv) other components utilized with the system (e.g., conductive material, nonconductive material, a soothing gel or cream, a bandage, etc.). The nature of these additional component(s)/reagent(s) will depend upon the particular treatment format, and identification thereof is well within the skill of one of ordinary skill in the art; therefore, no further description thereof is deemed necessary. Also, the components/reagents present in the kits may each be in separate containers/compartments, or various components/reagents can be combined in one or more containers/compartments, depending on the sterility, cross-reactivity, and stability of the components/reagents.

The kit may be disposed in any packaging that allows the components present therein to function in accordance with the present disclosure. In certain non-limiting embodiments, the kit further comprises a sealed packaging in which the components are disposed. In certain particular (but non-limiting) embodiments, the sealed packaging is substantially impermeable to air and/or substantially impermeable to light.

In addition, the kit can further include a set of written instructions explaining how to use one or more components of the kit. A kit of this nature can be used in any of the methods described or otherwise contemplated herein.

In certain non-limiting embodiments, the kit has a shelf life of at least about six months, such as (but not limited to), at least about nine months, or at least about 12 months.

Certain non-limiting embodiments of the present disclosure are related to systems that include any of the components of the alternating electric field generating systems disclosed or otherwise contemplated herein (such as, but not limited to, one or more transducer arrays and/or one or more hydrogel compositions, as disclosed in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016) in combination with at least one of any of the compositions disclosed or otherwise contemplated herein. The systems may optionally further include one or more of any of the optional compositions disclosed or otherwise contemplated herein. The systems may optionally further include one or more devices (or one or more components of devices) utilized in one or more additional therapy steps.

### EXAMPLES

Examples are provided herein below. However, the present disclosure is to be understood to not be limited in its application to the specific experimentation, results, and laboratory procedures disclosed herein after. Rather, the Examples are simply provided as one of various embodiments and is meant to be exemplary, not exhaustive.

### Example 1

Ovarian cancer is among the top 10 most deadly cancers among women globally, with a 4.0 per 100,000 age-standardized mortality rate in 2022. Nearly 20-30% of patients with high-grade serous ovarian cancer will have primary resistance to platinum agents; those who are sensitive will most likely develop recurrence and acquire progressive resistance over time.

Treatment options for Platinum-Resistant Ovarian Cancer (PROC) include systemic chemotherapies alone (i.e., paclitaxel, gemcitabine, doxorubicin formulations, and topotecan) or in combination with bevacizumab, and mirvetuximab soravtansine-gynx. However, PROC prognosis remains poor (median overall survival (OS) around 10-12 months), despite available treatment options. Therefore, a strong need for further innovations for well-tolerated and effective treatment persists.

TTFields therapy delivers electric fields which disrupt cellular processes critical for cancer cell viability and tumor progression. TTFields exert anti-mitotic effects through disruption of microtubules and enhance effects of taxanes, which target tubulin and induce cell cycle arrest. In addition, TTFields application together with paclitaxel demonstrated efficacy in preclinical ovarian models (Voloshin et al. (2016) Int J Cancer, 139 (12): 2850-2858). TTFields therapy has multiple regulatory approvals worldwide for glioblastoma and pleural mesothelioma, and has established efficacy in metastatic non-small cell lung cancer, with favorable safety. TTFields is a noninvasive locoregional treatment modality, delivered to the abdomen by a portable medical device and two pairs of arrays. Substantially continuous use (≥18 hours/day) of the system is recommended.

The pilot INNOVATE study (NCT02244502) demonstrated safety and feasibility of TTFields therapy with weekly paclitaxel in 31 patients with recurrent ovarian carcinoma (Vergote et al. (2018) Gynecol Oncol, 150 (3): 471-477). Median PFS was 8.9 months, and median OS was not reached; the 1-year survival rate was 61%.

TTFields' anti-mitotic effects are dose-dependent and may be affected by tissue conductivity changes. Preclinical data (unpublished) showed doxorubicin induces tumor fibrosis and changes tissue conductivity.

This randomized phase 3 study investigated TTFields therapy with paclitaxel (TTFields+PTX) vs paclitaxel (PTX) in patients with platinum-resistant ovarian cancer (PROC).

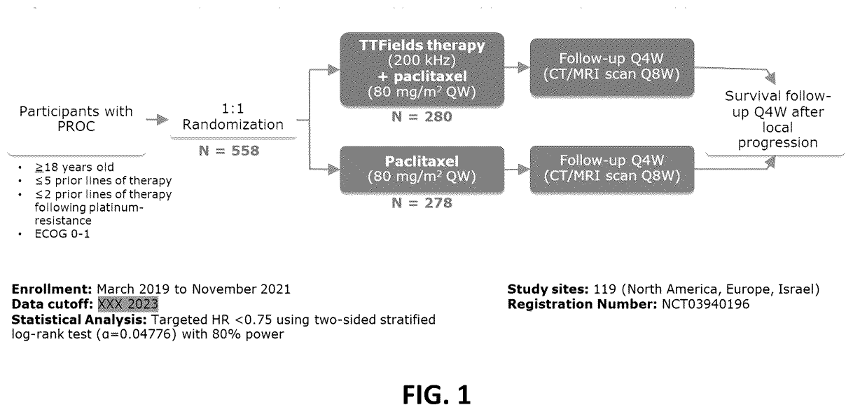

To evaluate the safety and efficacy of TTFields therapy with weekly paclitaxel compared to weekly paclitaxel alone in PROC, the Phase 3 study design shown in FIG. 1 was utilized. ENGOT-ov50/GOG-3029/INNOVATE-3 (NCT03940196) enrolled adults with PROC, ≤5 prior lines of therapy (LOT), ≤2 prior LOT following platinum-resistance, and ECOG 0-1. Patients received TTFields (200 kHz; ≥18 hours/day)+PTX (80 mg/m²) weekly or PTX. Primary endpoint was overall survival (OS). 558 patients were randomized to receive TTFields+PTX (n=280) or PTX (n=278) March 2019 to November 2021.

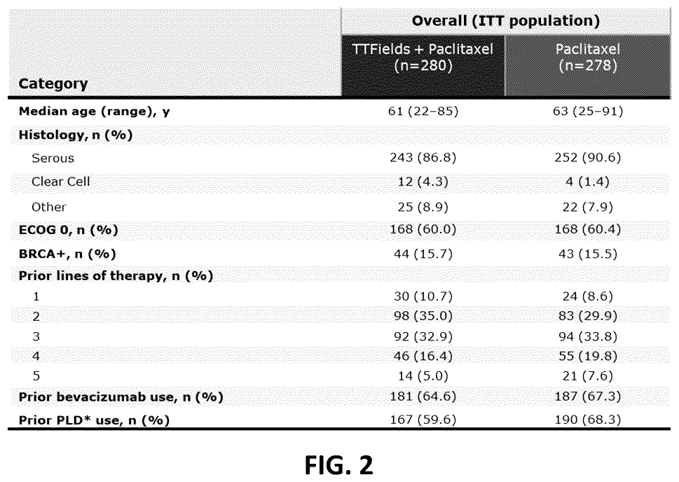

As shown in FIG. 2, baseline characteristics were well-balanced across both arms of the study (median age, 62 [22-91] years; serous histology, 88.7%; ECOG 0, 60.2%; BRCA+, 15.6%). Patients were heavily pretreated in the ITT population, as 24.4% of patients had received 4+prior lines of therapy (LOT), and 65.9% had received prior bevacizumab. In the overall intention-to-treat population, TTFields therapy median duration was 15.9 (0.1-159.7) weeks.

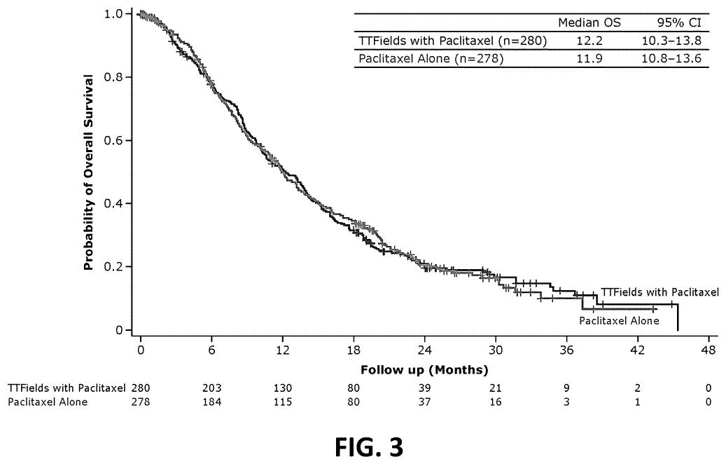

As shown in FIG. 3, median overall survival (OS) was 12.2 months with TTFields plus Paclitaxel (TTFields+PTX) versus 11.9 months with Paclitaxel (PTX) alone (HR, 1.01; 95% CI, 0.83-1.24; p=0.89). Therefore, when the overall cohort was examined, treatment with TTFields plus Paclitaxel did not significantly extend survival when compared to Paclitaxel alone.

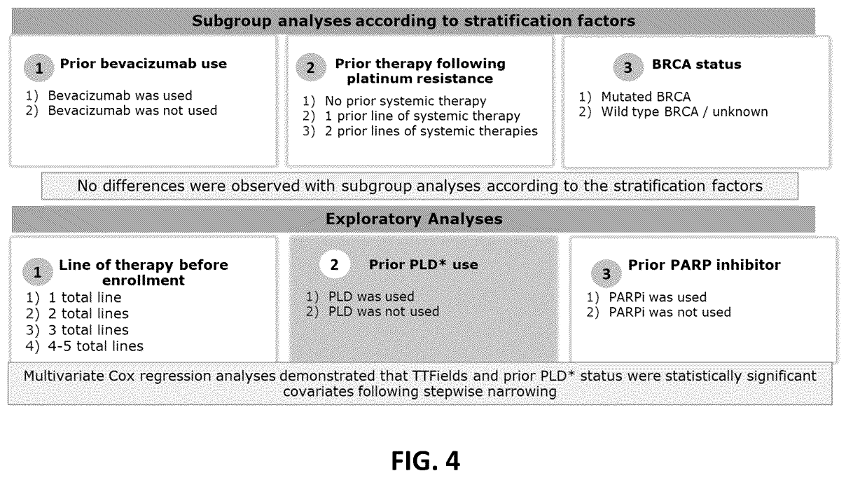

Therefore, subgroup and exploratory analyses were performed. FIG. 4 provides a summary of subgroup analyses according to stratification factors and exploratory analyses. No differences were observed with subgroup analyses according to the stratification factors. However, Multivariate Cox regression analyses to eliminate alternative covariates demonstrated that TTFields and prior PLD* status were statistically significant covariates following stepwise narrowing.

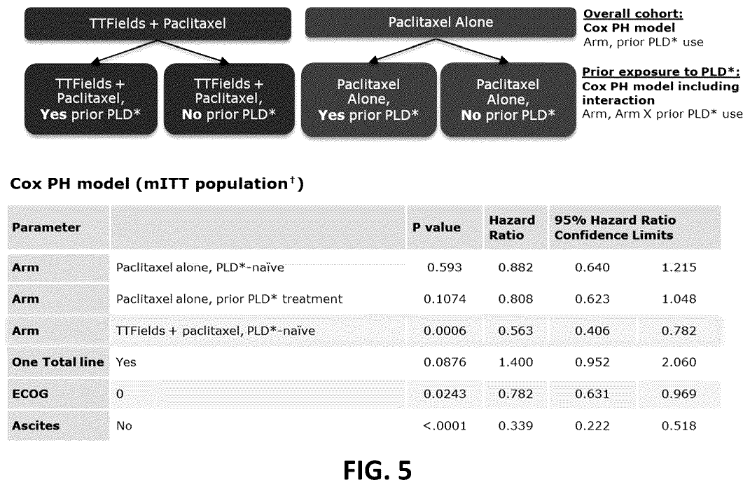

In addition, a multivariant analysis was performed, as shown in FIG. 5. Model parameters evaluated included Arm, Arm X prior PLD* use, prior bevacizumab, BRCA status, number of line following platinum, total number of prior lines, prior PARP, FIGO stage, grade, ascites, platinum free interval, patient age, and ECOG. Indicating variables maintained in the model defined by significance of 0.25 for variables exclusion. The inclusion of the interaction between treatment arm and prior PLD* use led to significant results for the treatment arm.

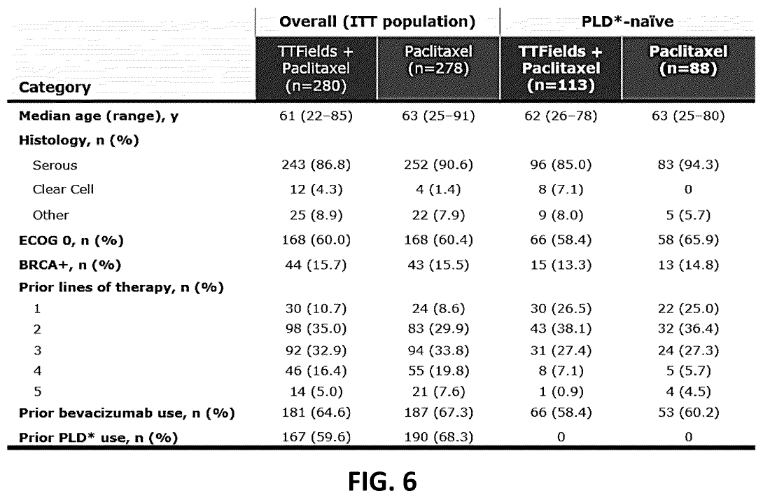

Based on these analyses, an analysis of the PLD*-naïve subgroup was performed. FIG. 6 lists the demographics and baseline characteristics of the PLD*-naïve subgroup compared to the overall ITT population. The PLD*-naïve subgroup represented 36% of the ITT population.

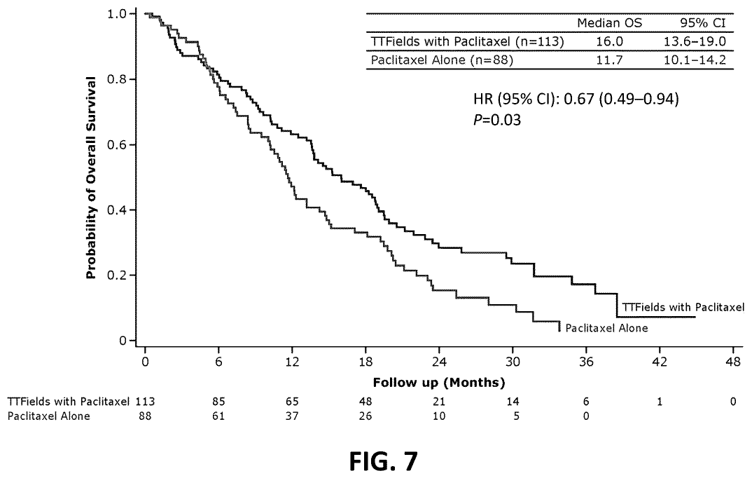

FIG. 7 depicts the overall survival in the PLD*-naïve subgroup. Post-hoc analysis in pegylated liposomal doxorubicin (PLD)-naïve patients showed extended survival with TTFields+PTX (n=113) vs PTX (n=88) (16 vs 11.7 months; HR, 0.67; 95% CI, 0.49-0.94; p=0.03). Therefore, a significantly improved survival rate was seen in the PLD*-naïve subgroup when compared to the overall cohort (which exhibited 12.2 months with TTFields plus Paclitaxel (TTFields+PTX) versus 11.9 months with Paclitaxel (PTX) alone, FIG. 3).

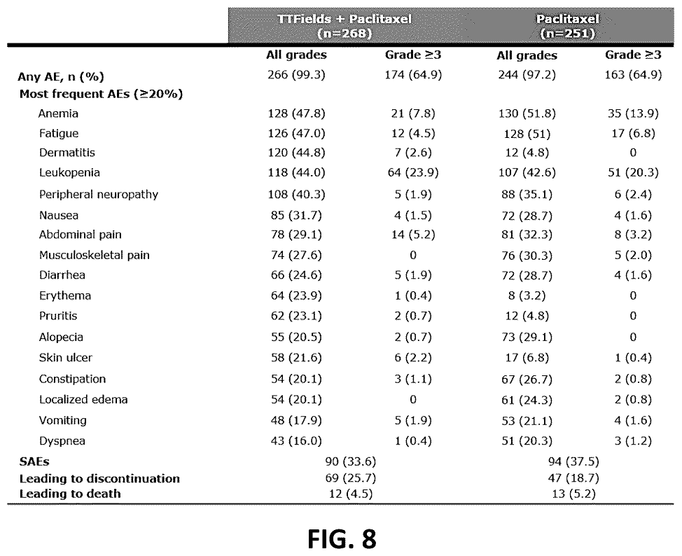

In addition, a safety analysis was also performed with respect to adverse events (AEs), as shown in FIG. 8. Similar incidence of grade ≥3 AEs were observed between the two treatment arms. Grade ≥3 adverse events (AEs) occurred in 60% of patients, with no TTFields-related increase in severe systemic toxicities; 83.6% of patients receiving TTFields had a device-related skin AE (all Grade 1/2). The most common grade ≥3 AE was leukopenia, which was similar between both arms. TTFields did not increase systemic AEs relative to paclitaxel alone. No new safety signals were identified in the PLD*-naïve subgroup (data not shown).

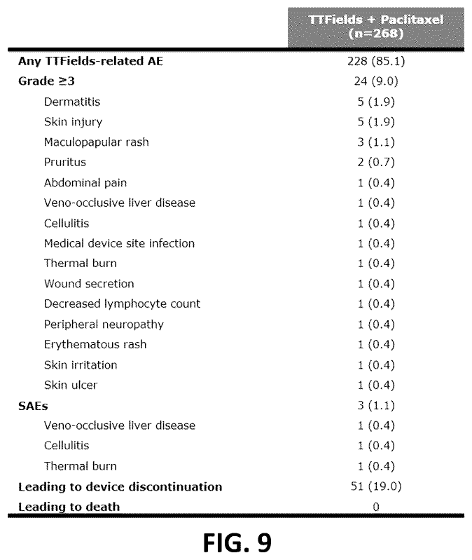

In addition, a safety analysis with respect to AEs specifically related to TTFields therapy was also undertaken, as shown in FIG. 9. Grade 1/2 device-related skin AEs occurred in 83.6% of patients who received TTFields therapy. There were no grade 4 AEs or deaths attributed to TTFields therapy. In addition, no new safety signals were identified in the PLD*-naïve subgroup (data not shown).

### CONCLUSION

In the overall cohort, TTFields+PTX did not improve OS compared with PTX. However, post-hoc analyses demonstrated clinically and statistically significant improvements in survival with TTFields therapy in PLD*-naïve patients with PROC, which represented one-third of the ITT population. TTFields therapy in patients with PROC was well-tolerated with no additive systemic toxicity and no new safety signals. The findings demonstrate therapeutic value of TTFields therapy in PLD-naïve patients with PROC.

### Example 2

Innovate-3 study excluding patients that had received doxorubicin prior to TTFields+Paclitaxel or Paclitaxel

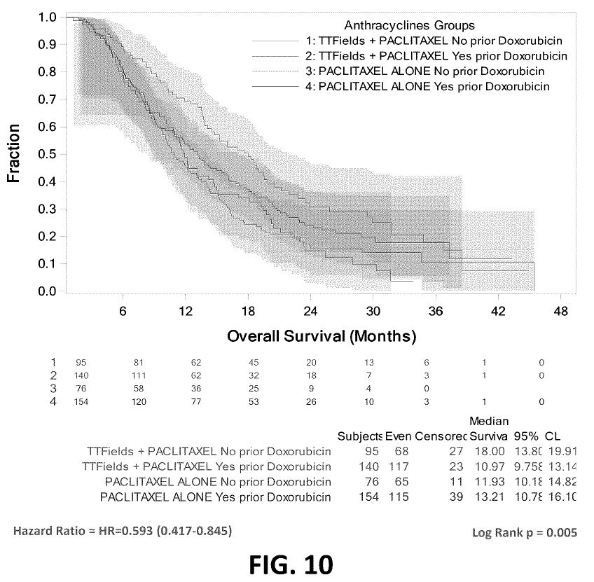

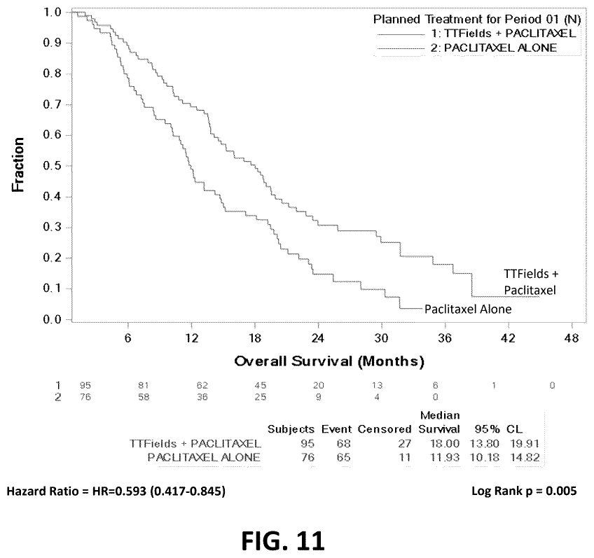

This Example contains further data related to the study of Example 1. FIGS. 10-11 include data of PROC patients that had received doxorubicin prior to treatment or were Doxorubicin-/PLD-naïve and which received treatment comprising TTFields plus Paclitaxel or Paclitaxel alone. With Doxorubicin treatment prior to use, overall survival was 10.97 months with TTFields and 13.21 months without TTFields. In contrast, the Doxorubicin-/PLD-naïve group without prior use of doxorubicin exhibited an overall survival of 18 months with TTFields and 11.93 months without TTFields. In addition, without prior use of doxorubicin/PLD, the hazard ratio for TTFields was 0.593 with a p value of 0.005.

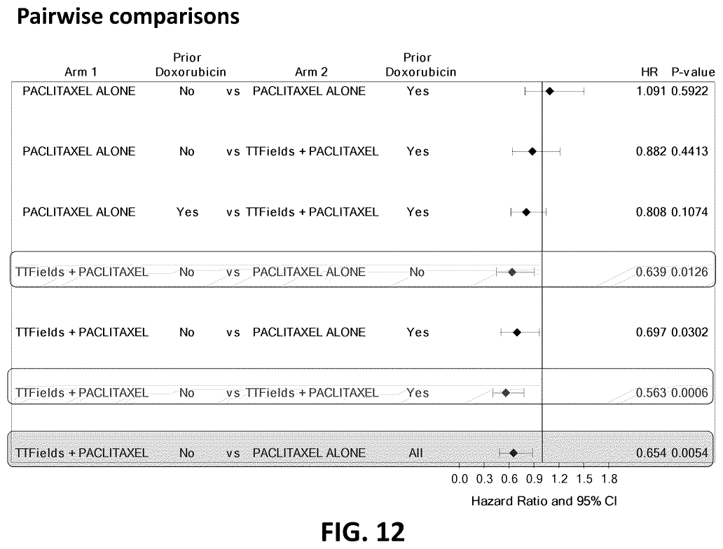

FIG. 12 illustrates a multivariate analysis for this Example. As shown, PLD-naïve patients had a 36% risk reduction for death in TTFields+Paclitaxel compared to Paclitaxel alone. In addition, PLD-naïve patients had a 44% risk reduction for death compared to patients that received prior PLD, when treated with TTFields.

### Non-Limiting Illustrative Embodiments of the Inventive Concept(s)

Illustrative embodiment 1. A method of treating cancer in a subject, the method comprising the steps of: (1) selecting a subject that is naïve for at least one standard-of-care chemotherapeutic agent for the cancer to be treated; and (2) applying an alternating electric field to a target region of the subject for a period of time.

Illustrative embodiment 2. A method of reducing a volume of a tumor present in a body of a living subject, wherein the tumor includes a plurality of cancer cells, the method comprising the steps of: (1) selecting a subject that is naïve for at least one standard-of-care chemotherapeutic agent for the cancer to be treated; and (2) applying an alternating electric field to a target region of the subject for a period of time.

Illustrative embodiment 3. A method of preventing an increase of volume of a tumor, wherein the tumor is present in a body of a living subject and includes a plurality of cancer cells, the method comprising the steps of: (1) selecting a subject that is naïve for at least one standard-of-care chemotherapeutic agent for the cancer to be treated; and (2) applying an alternating electric field to a target region of the subject for a period of time.

Illustrative embodiment 3A. The method of any of illustrative embodiments 1-3, wherein the at least one standard-of-care chemotherapeutic agent for which the subject is naïve is further defined as at least one profibrotic chemotherapeutic agent.

Illustrative embodiment 3B. The method of any of illustrative embodiments 1-3, wherein the at least one standard-of-care chemotherapeutic agent for which the subject is naïve is further defined as at least one anti-fibrotic chemotherapeutic agent.

Illustrative embodiment 3C. The method of any of illustrative embodiments 1-3, wherein the at least one standard-of-care chemotherapeutic agent for which the subject is naïve is further defined at least one non-fibrotic chemotherapeutic agent or at least one chemotherapeutic agent for which a fibrosis status is unknown.

Illustrative embodiment 3D. The method of any of illustrative embodiments 1-3, wherein the subject is naïve for all current standard-of-care chemotherapeutic agents for the cancer to be treated.

Illustrative embodiment 4. A method of treating cancer in a subject, the method comprising the steps of: (1) selecting a subject that is naïve for profibrotic chemotherapy and profibrotic antitumor treatment for the cancer to be treated; and (2) applying an alternating electric field to a target region of the subject for a period of time.

Illustrative embodiment 5. A method of reducing a volume of a tumor present in a body of a living subject, wherein the tumor includes a plurality of cancer cells, the method comprising the steps of: (1) selecting a subject that is naïve for profibrotic chemotherapy and profibrotic antitumor treatment for the cancer to be treated; and (2) applying an alternating electric field to a target region of the subject for a period of time.

Illustrative embodiment 6. A method of preventing an increase of volume of a tumor, wherein the tumor is present in a body of a living subject and includes a plurality of cancer cells, the method comprising the steps of: (1) selecting a subject that is naïve for profibrotic chemotherapy and profibrotic antitumor treatment for the cancer to be treated; and (2) applying an alternating electric field to a target region of the subject for a period of time.

Illustrative embodiment 7. The method of any of illustrative embodiments 1-6, further comprising the step of: (3) administering at least one composition to the subject, wherein the at least one composition comprises at least one chemotherapeutic agent.

Illustrative embodiment 7A. The method of illustrative embodiment 7, wherein the at least one composition administered to the subject in step (3) comprises at least one anti-fibrotic chemotherapeutic agent.

Illustrative embodiment 7B. The method of illustrative embodiment 7, wherein the at least one composition administered to the subject in step (3) comprises at least one non-fibrotic chemotherapeutic agent.

Illustrative embodiment 7C. The method of illustrative embodiment 7, wherein the at least one composition administered to the subject in step (3) comprises at least one profibrotic chemotherapeutic agent.

Illustrative embodiment 8. The method of any of illustrative embodiments 1-7C, wherein the subject has glioblastoma, and wherein the subject is naïve for at least one standard-of-care chemotherapeutic agent selected from the group consisting of temozolomide (TMZ), bevacizumab, carmustine, and combinations thereof.

Illustrative embodiment 8A. The method of illustrative embodiment 8, wherein the subject is naïve for at least two of temozolomide (TMZ), bevacizumab, and Carmustine.

Illustrative embodiment 8B. The method of illustrative embodiment 8, wherein the subject is naïve for temozolomide (TMZ), bevacizumab, and Carmustine.

Illustrative embodiment 9. The method of any of illustrative embodiments 1-8B, wherein the subject has mesothelioma, and wherein the subject is naïve for at least one standard-of-care chemotherapeutic agent selected from the group consisting of bevacizumab, carboplatin, cisplatin, cyclophophamide, doxorubicin, gemcitabine, ipilimumab, nivolumab, oxaliplatin, pembrolizumab, pemetrexed, raltitrexed, vinorelbine, and combinations thereof.

Illustrative embodiment 9A. The method of illustrative embodiment 9, wherein the subject is naïve for at least two, at least three, at least four, at least five, at least six, at least seven, at least eight, at least nine, at least ten, at least eleven, or at least twelve of bevacizumab, carboplatin, cisplatin, cyclophophamide, doxorubicin, gemcitabine, ipilimumab, nivolumab, oxaliplatin, pembrolizumab, pemetrexed, raltitrexed, and vinorelbine.

Illustrative embodiment 9B. The method of illustrative embodiment 9, wherein the subject is naïve for all of bevacizumab, carboplatin, cisplatin, cyclophophamide, doxorubicin, gemcitabine, ipilimumab, nivolumab, oxaliplatin, pembrolizumab, pemetrexed, raltitrexed, and vinorelbine.

Illustrative embodiment 10. The method of any of illustrative embodiments 1-9B, wherein the subject has ovarian cancer, and wherein the subject is naïve for at least one standard-of-care chemotherapeutic agent selected from the group consisting of docetaxel, gemcitabine, paclitaxel, PARP Inhibitors, and combinations thereof.

Illustrative embodiment 10A. The method of illustrative embodiment 10, wherein the subject is naïve for at least two or at least three of docetaxel, gemcitabine, paclitaxel, and PARP Inhibitors.

Illustrative embodiment 10B. The method of illustrative embodiment 10, wherein the subject is naïve for all of docetaxel, gemcitabine, paclitaxel, and PARP Inhibitors.

Illustrative embodiment 11. The method of any of illustrative embodiments 1-10B, wherein the subject has pancreatic cancer, and wherein the subject is naïve for at least one standard-of-care chemotherapeutic agent selected from the group consisting of capecitabine, fluorouracil, folfirinox, gemcitabine, irinotecan, leucovorin, nalirifox, oxaliplatin, paclitaxel, pemetrexed, and combinations thereof.

Illustrative embodiment 11A. The method of illustrative embodiment 11, wherein the subject is naïve for at least two, at least three, at least four, at least five, at least six, at least seven, at least eight, or at least nine of capecitabine, fluorouracil, folfirinox, gemcitabine, irinotecan, leucovorin, nalirifox, oxaliplatin, paclitaxel, and pemetrexed.

Illustrative embodiment 11B. The method of illustrative embodiment 11, wherein the subject is naïve for all of capecitabine, fluorouracil, folfirinox, gemcitabine, irinotecan, leucovorin, nalirifox, oxaliplatin, paclitaxel, and pemetrexed.

Illustrative embodiment 12. The method of any of illustrative embodiments 1-11B, wherein the subject has non-small cell lung cancer, and wherein the subject is naïve for at least one standard-of-care chemotherapeutic agent selected from the group consisting of carboplatin, cisplatin, docetaxel, etoposide, gemcitabine, paclitaxel, pemetrexed, vinorelbine, and combinations thereof.

Illustrative embodiment 12A. The method of illustrative embodiment 12, wherein the subject is naïve for at least two, at least three, at least four, at least five, at least six, or at least seven of carboplatin, cisplatin, docetaxel, etoposide, gemcitabine, paclitaxel, pemetrexed, and vinorelbine.

Illustrative embodiment 12B. The method of illustrative embodiment 12, wherein the subject is naïve for all of carboplatin, cisplatin, docetaxel, etoposide, gemcitabine, paclitaxel, pemetrexed, and vinorelbine.

Illustrative embodiment 13. A method of treating cancer in a subject, the method comprising the steps of: (1) selecting a subject that is doxorubicin-naïve; and (2) applying an alternating electric field to a target region of the subject for a period of time.

Illustrative embodiment 14. A method of reducing a volume of a tumor present in a body of a living subject, wherein the tumor includes a plurality of cancer cells, the method comprising the steps of: (1) selecting a subject that is doxorubicin-naïve; and (2) applying an alternating electric field to a target region of the subject for a period of time.

Illustrative embodiment 15. A method of preventing an increase of volume of a tumor, wherein the tumor is present in a body of a living subject and includes a plurality of cancer cells, the method comprising the steps of: (1) selecting a subject that is doxorubicin-naïve; and (2) applying an alternating electric field to a target region of the subject for a period of time.

Illustrative embodiment 16. The method of any of illustrative embodiments 13-15, further comprising the step of: (3) administering at least one composition to the subject, wherein the at least one composition comprises at least one chemotherapeutic agent.

Illustrative embodiment 17. The method of any of illustrative embodiments 1-16, wherein at least one of: the alternating electric field is applied at a frequency in a range of from about 50 kHz to about 1 MHz; the alternating electric field has a field strength of at least about 1 V/cm in at least a portion of the cancer cells/target region of the subject; and the period of time that the alternating electric field is applied is at least about 50% of a 24 consecutive hour time period (i.e., at least about 12 cumulative hours of a 24 hour period).

Illustrative embodiment 18. The method of any one of illustrative embodiments 1-17, wherein administration of the at least one composition and initiation of application of the alternating electric field are performed wholly or partially simultaneously.

Illustrative embodiment 19. The method of any one of illustrative embodiments 1-18, wherein the at least one composition is administered after application of the alternating electric field has begun.

Illustrative embodiment 20. The method of illustrative embodiment 19, wherein the at least one composition is administered before the period of time that the alternating electric field is applied has elapsed.

Illustrative embodiment 21. The method of illustrative embodiment 19, wherein the at least one composition is administered after the period of time has elapsed.

Illustrative embodiment 22. The method of any one of illustrative embodiments 1-21, wherein one or more of the steps is repeated one or more times.

Illustrative embodiment 23. The method of any one of illustrative embodiments 1-22, wherein the cancer is in the form of at least one solid tumor.

Illustrative embodiment 24. The method of any one of illustrative embodiments 1-23, wherein the at least one chemotherapeutic agent is selected from the group consisting of paclitaxel, nimustine, gefitinib, pazopanib, nintedanib, sorafenib, sunitinib, imatinib, everolimus, temozolomide, docetaxel, lomustine, pemetrexed, vinorelbine, irinotecan, etoposide, afatinib, osimertinib, erlotinib, pazopanib, sotorasinib, ramucirumab, mirvetuximab, necitumumab, niraparib, durvalumab, atezolizumab, cemiplimab, avelumab, and combinations thereof.

Illustrative embodiment 25. The method of any one of illustrative embodiments 1-24, optionally wherein the composition administered in step (3) (when present) further comprises at least one anti-fibrotic agent.

Illustrative embodiment 26. The method of any one of illustrative embodiments 1-25, wherein the method further comprises the step of: (4) administering at least one additional composition to the subject, wherein the at least one additional composition comprises at least one anti-fibrotic agent.

Illustrative embodiment 27. The method of illustrative embodiment 26, wherein steps (3) and (4) are performed simultaneously.

Illustrative embodiment 28. The method of illustrative embodiment 26, wherein steps (3) and (4) are performed wholly or partially sequentially.

Illustrative embodiment 29. The method of any one of illustrative embodiments 25-28, wherein the at least one anti-fibrotic agent is selected from the group consisting of a calcium channel blocker, an angiotensin II receptor blocker (ARB), an IL11 inhibitor, an IL13 inhibitor, a receptor tyrosine kinase inhibitor (RTKI), a rennin-angiotensin-aldosterone system (RAAS) inhibitor, an angiotensin-converting enzyme (ACE) inhibitor, an anti-hypertension agent, and combinations thereof.

Illustrative embodiment 30. The method of any one of illustrative embodiments 25-29, wherein the at least one anti-fibrotic agent is selected from the group consisting of losartan, fasudil, pirfenidone, pintedanib, nintedanib, hyaluronidase, tranilast, vismodegib, felodipine, verapamil, diltiazem, nifedipine, and combinations thereof.

Illustrative embodiment 31. The method of any one of illustrative embodiments 25-30, optionally wherein the at least one chemotherapeutic agent administered in step (3) (when present) comprises at least one anthracycline.

Illustrative embodiment 32. The method of any one of illustrative embodiments 25-31, optionally wherein the at least one chemotherapeutic agent administered in step (3) (when present) is selected from the group consisting of paclitaxel, nimustine, gefitinib, pazopanib, nintedanib, sorafenib, sunitinib, imatinib, everolimus, temozolomide, docetaxel, lomustine, pemetrexed, raltitrexed, vinorelbine, irinotecan, etoposide, afatinib, osimertinib, erlotinib, pazopanib, sotorasinib, ramucirumab, mirvetuximab, necitumumab, niraparib, durvalumab, atezolizumab, cemiplimab, avelumab, Daunorubicin, Doxorubicin, Epirubicin, Idarubicin, Mitotaxantrone, Valrubicin, bleomycin, carmustine, doxorubicin, cisplatin, oxaliplatin, gemcitabine, cyclophosphamide, fluorouracil, leucovorin, capecitabine, mitoxantrone, bevacizumab, carboplatin, olaparib, dacomitinib, and combinations thereof.

Illustrative embodiment 33. The method of any one of illustrative embodiments 1-32, wherein at least one of steps (2) and (3) (and step (4), if present) is repeated one or more times.

Illustrative embodiment 34. The method of any one of illustrative embodiments 1-33, wherein the cancer is selected from the group consisting of hepatocellular carcinoma, glioblastoma, pleural mesothelioma, differentiated thyroid cancer, advanced renal cell carcinoma, ovarian cancer, pancreatic cancer, lung cancer cell, breast cancer, colorectal cancer, and combinations thereof.

Illustrative embodiment 35. The method of illustrative embodiment 34, wherein the cancer is ovarian cancer, and optionally wherein the at least one chemotherapeutic agent administered in step (3) (when present) comprises paclitaxel.

Illustrative embodiment 36. The method of any one of illustrative embodiments 1-35, wherein the cancer to be treated is selected from the group consisting of soft tissue cancer, bone cancer, breast cancer, ovarian cancer, bladder cancer, thyroid cancer, and small cell lung cancer; wherein the at least one standard-of-care chemotherapeutic agent for which the subject is naïve comprises doxorubicin, and optionally wherein the at least one chemotherapeutic agent administered in step (3) (when present) comprises paclitaxel.

Illustrative embodiment 37. The method of any one of illustrative embodiments 1-36, wherein the cancer to be treated is ovarian cancer, wherein the at least one standard-of-care chemotherapeutic agent for which the subject is naïve comprises doxorubicin, and optionally wherein the at least one chemotherapeutic agent administered in step (3) (when present) comprises paclitaxel.

Illustrative embodiment 38. The method of any one of illustrative embodiments 1-37, further defined as a method of reducing a volume of a tumor, wherein the tumor is present in a body of a living subject and includes a plurality of cancer cells.

Illustrative embodiment 39. The method of any one of illustrative embodiments 1-38, further defined as a method of preventing an increase of volume of the tumor, wherein the tumor is present in a body of a living subject and includes a plurality of cancer cells.

Illustrative embodiment 40. The method of any one of illustrative embodiments 1-39, wherein the method is performed in the absence of step (3) (i.e., an alternating electric field monotherapy method).

Illustrative embodiment 41. A composition comprising at least one chemotherapeutic agent for use in the method of any of illustrative embodiments 1-40.

Illustrative embodiment 41A. The composition of illustrative embodiment 41, wherein the at least one chemotherapeutic agent is further defined as at least one anti-fibrotic chemotherapeutic agent.

Illustrative embodiment 41B. The composition of illustrative embodiment 41, wherein the at least one chemotherapeutic agent is further defined at least one profibrotic (or likely profibrotic) chemotherapeutic agent.

Illustrative embodiment 41C. The composition of illustrative embodiment 41, wherein the at least one chemotherapeutic agent is further defined at least one non-fibrotic chemotherapeutic agent or a chemotherapeutic agent for which a fibrosis status is unknown.

Illustrative embodiment 42. The composition of any of illustrative embodiments 41-41C, further comprising at least one anti-fibrotic agent.

Illustrative embodiment 43. A combination treatment comprising an alternating electric field and at least one composition comprising at least one chemotherapeutic agent, for use in the method of any of illustrative embodiments 1-40.

Illustrative embodiment 43A. The combination treatment of illustrative embodiment 43, wherein the at least one chemotherapeutic agent is further defined as at least one anti-fibrotic chemotherapeutic agent.

Illustrative embodiment 43B. The combination treatment of illustrative embodiment 43, wherein the at least one chemotherapeutic agent is further defined at least one profibrotic (or likely profibrotic) chemotherapeutic agent.

Illustrative embodiment 43C. The combination treatment of illustrative embodiment 43, wherein the at least one chemotherapeutic agent is further defined at least one non-fibrotic chemotherapeutic agent or a chemotherapeutic agent for which a fibrosis status is unknown.

Illustrative embodiment 44. The combination treatment of any of illustrative embodiments 43-43C, further comprising at least one additional composition, wherein the at least one additional composition comprises at least one anti-fibrotic agent.

Illustrative embodiment 45. A kit for use in the method of any of illustrative embodiments 1-40, the kit comprising: an alternating electric field-generating device; and at least one composition comprising at least one chemotherapeutic agent.

Illustrative embodiment 45A. The kit of illustrative embodiment 45, wherein the at least one chemotherapeutic agent is further defined as at least one anti-fibrotic chemotherapeutic agent.

Illustrative embodiment 45B. The kit of illustrative embodiment 45, wherein the at least one chemotherapeutic agent is further defined at least one profibrotic (or likely profibrotic) chemotherapeutic agent.

Illustrative embodiment 45C. The kit of illustrative embodiment 45, wherein the at least one chemotherapeutic agent is further defined at least one non-fibrotic chemotherapeutic agent or a chemotherapeutic agent for which a fibrosis status is unknown.

Illustrative embodiment 46. The kit of any of illustrative embodiments 45-45C, further comprising at least one additional composition, wherein the at least one additional composition comprises at least one anti-fibrotic agent.

Illustrative embodiment 47. A system for use in the method of any of illustrative embodiments 1-40, the system comprising: an alternating electric field-generating device; and at least one composition comprising at least one chemotherapeutic agent.

Illustrative embodiment 47A. The system of illustrative embodiment 47, wherein the at least one chemotherapeutic agent is further defined as at least one anti-fibrotic chemotherapeutic agent.

Illustrative embodiment 47B. The system of illustrative embodiment 47, wherein the at least one chemotherapeutic agent is further defined at least one profibrotic (or likely profibrotic) chemotherapeutic agent.

Illustrative embodiment 47C. The system of illustrative embodiment 47, wherein the at least one chemotherapeutic agent is further defined at least one non-fibrotic chemotherapeutic agent or a chemotherapeutic agent for which a fibrosis status is unknown.

Illustrative embodiment 48. The system of any of illustrative embodiments 47-47C, further comprising at least one additional composition, wherein the at least one additional composition comprises at least one anti-fibrotic agent.

Illustrative embodiment 49. The composition/combination treatment/kit/system of any of illustrative embodiments 42, 44, 46, or 48, wherein the at least one anti-fibrotic agent is selected from the group consisting of a calcium channel blocker, an angiotensin II receptor blocker (ARB), an IL11 inhibitor, an IL13 inhibitor, a receptor tyrosine kinase inhibitor (RTKI), a rennin-angiotensin-aldosterone system (RAAS) inhibitor, an angiotensin-converting enzyme (ACE) inhibitor, an anti-hypertension agent, and combinations thereof.

Illustrative embodiment 50. The composition/combination treatment/kit/system of any of illustrative embodiments 41A, 43A, 45A, or 47A, wherein the anti-fibrotic chemotherapeutic agent is selected from the group consisting of a taxane, paclitaxel, docetaxel, cabazitaxel, abraxane, nimustine, gefitinib, pazopanib, nintedanib, sorafenib, sunitinib, imatinib, everolimus, and combinations thereof.

Illustrative embodiment 51. The composition/combination treatment/kit/system of any of illustrative embodiments 41B, 43B, 45B, or 47B, wherein the profibrotic (or likely profibrotic) chemotherapeutic agent is selected from the group consisting of an anthracycline, Daunorubicin, Doxorubicin, Epirubicin, Idarubicin, Mitotaxantrone, Valrubicin, bleomycin, carmustine, cisplatin, oxaliplatin, gemcitabine, cyclophosphamide, fluorouracil, leucovorin, capecitabine, mitoxantrone, bevacizumab, carboplatin, olaparib, dacomitinib, raltitrexed, and combinations thereof.

Illustrative embodiment 52. The composition/combination treatment/kit/system of any of illustrative embodiments 41C, 43C, 45C, or 47C, wherein the chemotherapeutic agent is selected from the group consisting of temozolomide, docetaxel, lomustine, pemetrexed, vinorelbine, irinotecan, etoposide, afatinib, osimertinib, erlotinib, pazopanib, sotorasinib, ramucirumab, mirvetuximab, necitumumab, niraparib, durvalumab, atezolizumab, cemiplimab, avelumab, and combinations thereof.

While the attached disclosures describe the inventive concept(s) in conjunction with the specific experimentation, results, and language set forth hereinafter, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A method of treating cancer in a subject, the method comprising the steps of:
(1) selecting a subject that is naïve for at least one standard-of-care chemotherapeutic agent for the cancer to be treated, wherein the cancer is in the form of at least one solid tumor;
(2) applying an alternating electric field to a target region of the subject for a period of time; and
(3) administering at least one composition to the subject, wherein the at least one composition comprises at least one chemotherapeutic agent.

2. The method of claim 1, wherein at least one of:
(i) the cancer to be treated is selected from the group consisting of soft tissue cancer, bone cancer, breast cancer, ovarian cancer, bladder cancer, thyroid cancer, and small cell lung cancer, and wherein the at least one standard-of-care chemotherapeutic agent for which the subject is naïve comprises doxorubicin;
(ii) wherein the cancer to be treated is glioblastoma, and wherein the subject is naïve for at least one standard-of-care chemotherapeutic agent selected from the group consisting of temozolomide (TMZ), bevacizumab, Carmustine, and combinations thereof;
(iii) wherein the cancer to be treated is mesothelioma, and wherein the subject is naïve for at least one standard-of-care chemotherapeutic agent selected from the group consisting of bevacizumab, carboplatin, cisplatin, cyclophophamide, doxorubicin, gemcitabine, ipilimumab, nivolumab, oxaliplatin, pembrolizumab, pemetrexed, raltitrexed, vinorelbine, and combinations thereof;
(iv) wherein the cancer to be treated is ovarian cancer, and wherein the subject is naïve for at least one standard-of-care chemotherapeutic agent selected from the group consisting of docetaxel, gemcitabine, paclitaxel, PARP Inhibitors, and combinations thereof;
(v) wherein the cancer to be treated is pancreatic cancer, and wherein the subject is naïve for at least one standard-of-care chemotherapeutic agent selected from the group consisting of capecitabine, fluorouracil, folfirinox, gemcitabine, irinotecan, leucovorin, nalirifox, oxaliplatin, paclitaxel, pemetrexed, and combinations thereof; or
(vi) wherein the cancer to be treated is non-small cell lung cancer, and wherein the subject is naïve for at least one standard-of-care chemotherapeutic agent selected from the group consisting of carboplatin, cisplatin, docetaxel, etoposide, gemcitabine, paclitaxel, pemetrexed, vinorelbine, and combinations thereof.

3. The method of claim 1, wherein the cancer to be treated is ovarian cancer, wherein the at least one standard-of-care chemotherapeutic agent for which the subject is naïve comprises doxorubicin, and wherein the at least one chemotherapeutic agent administered in step (3) comprises paclitaxel.

4. The method of claim 1, wherein at least one of:
the alternating electric field is applied at a frequency in a range of from about 50 kHz to about 1 MHz;
the alternating electric field has a field strength of at least about 1 V/cm in at least a portion of the target region of the subject; and
the period of time that the alternating electric field is applied is at least about 50% of a 24 consecutive hour time period.

5. The method of claim 1, wherein the at least one chemotherapeutic agent administered in step (3) is selected from the group consisting of paclitaxel, nimustine, gefitinib, pazopanib, nintedanib, sorafenib, sunitinib, imatinib, everolimus, temozolomide, docetaxel, and combinations thereof.

6. The method of claim 1, wherein the composition of (3) further comprises at least one anti-fibrotic agent.

7. The method of claim 1, wherein the method further comprises the step of:
(4) administering at least one additional composition to the subject, wherein the at least one additional composition comprises at least one anti-fibrotic agent.

8. The method of claim 7, wherein steps (3) and (4) are performed simultaneously.

9. The method of claim 7, wherein steps (3) and (4) are performed wholly or partially sequentially.

10. The method of claim 6, wherein the at least one anti-fibrotic agent is selected from the group consisting of a calcium channel blocker, an angiotensin II receptor blocker (ARB), an IL11 inhibitor, an IL13 inhibitor, a receptor tyrosine kinase inhibitor (RTKI), a rennin-angiotensin-aldosterone system (RAAS) inhibitor, an angiotensin-converting enzyme (ACE) inhibitor, an anti-hypertension agent, and combinations thereof.

11. The method of claim 1, wherein the cancer is selected from the group consisting of hepatocellular carcinoma, glioblastoma, pleural mesothelioma, differentiated thyroid cancer, advanced renal cell carcinoma, ovarian cancer, pancreatic cancer, lung cancer cell, breast cancer, colorectal cancer, and combinations thereof.

12. A method of treating cancer in a subject, the method comprising the steps of:
(1) selecting a subject that is naïve for profibrotic chemotherapy and profibrotic antitumor treatment for the cancer to be treated;
(2) applying an alternating electric field to a target region of the subject for a period of time; and
(3) administering at least one composition to the subject, wherein the at least one composition comprises at least one chemotherapeutic agent.

13. The method of claim 12, wherein at least one of:
the alternating electric field is applied at a frequency in a range of from about 50 kHz to about 1 MHz;
the alternating electric field has a field strength of at least about 1 V/cm in at least a portion of the target region of the subject; and
the period of time that the alternating electric field is applied is at least about 50% of a 24 consecutive hour time period.

14. The method of claim 12, wherein the at least one chemotherapeutic agent is selected from the group consisting of paclitaxel, nimustine, gefitinib, pazopanib, nintedanib, sorafenib, sunitinib, imatinib, everolimus, temozolomide, docetaxel, and combinations thereof.

15. The method of claim 12, wherein the composition of (3) further comprises at least one anti-fibrotic agent.

16. The method of claim 12, wherein the method further comprises the step of:
(4) administering at least one additional composition to the subject, wherein the at least one additional composition comprises at least one anti-fibrotic agent.

17. The method of claim 16, wherein steps (3) and (4) are performed simultaneously.

18. The method of claim 16, wherein steps (3) and (4) are performed wholly or partially sequentially.

19. The method of claim 12, wherein the cancer is selected from the group consisting of hepatocellular carcinoma, glioblastoma, pleural mesothelioma, differentiated thyroid cancer, advanced renal cell carcinoma, ovarian cancer, pancreatic cancer, lung cancer cell, breast cancer, colorectal cancer, and combinations thereof.

20. The method of claim 19, wherein the cancer is ovarian cancer, and wherein the at least one chemotherapeutic agent administered in step (3) comprises paclitaxel.
